(19) United States
(12) Patent Application Publication
(10) Pub. No.: US 2016/0088136 A1
(43) Pub. Date: Mar. 24, 2016

(54) Smartphone Based Meter and Injector

(71) Applicants: Paolo Di Donato, Hamilton (CA); John Finley, Welland (CA)

(72) Inventors: Paolo Di Donato, Hamilton (CA); John Finley, Welland (CA)

(21) Appl. No.: 14/495,509

(22) Filed: Sep. 24, 2014

(51) Int. Cl. H04M 1/21; A61B 5/151; A61M 5/178; A61B 5/157; A61B 5/00; H04W 4/00; A61B 5/145

(52) U.S. Cl. CPC H04M 1/21; H04W 4/008; A61B 5/151; A61B 5/14532; A61B 5/157; A61B 5/4839; A61B 5/6898; A61B 5/486; A61M 5/178

(57) ABSTRACT

One embodiment of the present invention incorporates a comprehensive blood glucose monitoring system into the case of a smartphone or other device that a patient normally carries with them. This system may include a glucose meter, lancets, test strips, logic to report the result of the blood test in the glucose meter, and a communication module to report the results to the smartphone. In addition, the system may also include small syringes of insulin concealed within the case or attached to the case. This would allow the user to inject if he or she needed to, after obtaining the results of the test.

### CROSS-REFERENCE TO RELATED APPLICATIONS

This application claims priority benefit of U.S. Provisional Patent Application No. 61/881,774, entitled “Smartphone Based Glucose Meter,” filed Sep. 24, 2013, and U.S. Provisional Patent Application No. 62/026,703, entitled “Smartphone Based Meter and Injector,” filed Jul. 21, 2014. The disclosures in those applications are incorporated herein in their entirety.

### BACKGROUND AND SUMMARY

The present invention relates generally to glucose meters, and more particularly, to a glucose meter and injector system that is incorporated into the structure of a smartphone or mobile electronic device.

The goal of diabetes care is to keep blood glucose levels as normal as possible in order to counteract the complications of diabetes. Complications include heart disease, high blood pressure, stroke, eye disease/blindness, kidney disease, foot disease, complications in pregnancy, and skin/dental disease. Diabetics require regular feedback regarding their current blood glucose levels, depending upon this feedback to provide guidance on how to improve future readings. It is this monitoring that influences their long term health.

Most diabetics use some type of glucose meter to regularly test their blood glucose throughout the day. The process of checking glucose levels with a typical meter occurs when blood is drawn, then placed on a disposable test strip and positioned in the meter. In one example of a glucose meter, the test strips are coated with chemicals, such as glucose oxidase, dehydrogenase, or hexokinase, that combines with glucose in the blood. The meter is then able to measure the glucose level present in the blood based on the reactions with these chemicals using a sensor.

Having a small, portable blood glucose monitoring system is a convenient way for diabetics to keep check and stay in tune with their condition. By incorporating a comprehensive glucose monitoring system into the structure or case of a smartphone or other device that a patient normally carries with them. Diabetics may recognize a significant improvement in terms of convenience and portability by having the device incorporated into another device. Importantly, one embodiment of the present invention will be capable of being incorporated into different commercially-available smartphone models on the market today and readily adaptable to any future models, thus providing a way to keep the monitor system close at hand at all times. In addition, the system may also include small syringes of insulin concealed within the case or attached to the case. This would allow the user to inject if he or she needed to, after obtaining the results of the test.

### BRIEF DESCRIPTION OF THE FIGURES

FIG. 1 shows a view of one embodiment of the glucose monitor smartphone case attached to the rear of a smartphone.

FIG. 2 shows one embodiment of the glucose monitor case with the rear cover of the case moved partially away from the case structure.

FIG. 3 shows a rear view of one embodiment of the glucose monitor case with the rear cover of the case moved partially away from the case structure, to reveal the internal space of the case looking towards the rear of the smartphone.

FIG. 4 shows an embodiment of the glucose monitor case wherein the lancet and glucose tester is contained in a single unit that is contained in the rear of the case.

FIG. 5 shows an embodiment where a glucose test insert is slid into the case structure to form the rear surface of the case.

### DETAILED DESCRIPTION

While the exemplary embodiments illustrated herein may show various features, it will be understood that the different features disclosed herein can be combined variously to achieve the objectives of the present invention.

Accordingly, one objective of the present invention is to have all necessary glucose monitoring apparatus a diabetic needs to complete a glucose level test held within a smartphone or electronic device case. A comprehensive encasement of all necessary components would include: a glucose meter, a lancer, lancets, test strips (finger strips), and the “integrator,” or means for transmitting the test results to the smartphone or electronic device. For the purposes of this application, any processor, logic circuit, communication means, or software may be referred to as a module.

The integrator may use wired electronic transmission from the glucose meter case to the audio jack, micro USB jack, or other connector to the smartphone. Or it may interface directly to a wired connection on the smartphone. For example, some smartphones provide connectors on their backside, and these connectors can be used to interface external devices to the smartphone. In another embodiment, it may use wireless transmission from the glucose meter case to the smartphone, such as Bluetooth or Wi-Fi, which relays glucose level data to the smartphone. This invention is intended to encompass any type of transmission of the data from the case to the smartphone known or later arising in the art; the specific means does not alter the spirit of the invention.

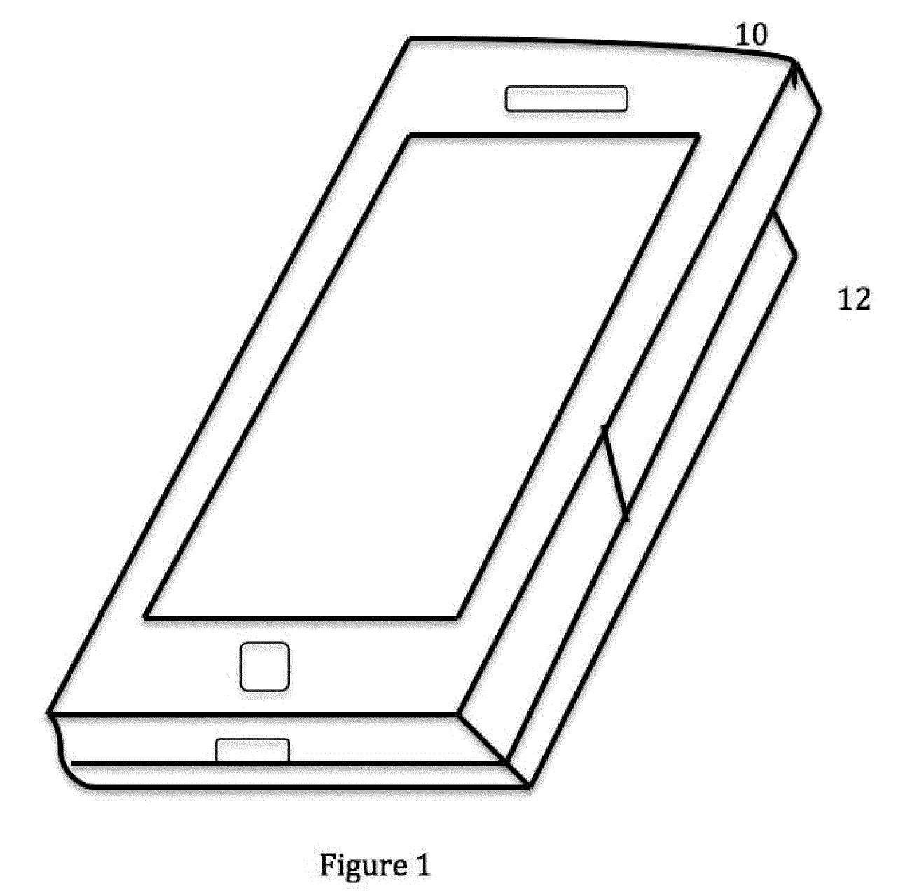

FIG. 1 shows one embodiment of the invention, the glucose meter serves a dual purpose of being a protective cover 12 for a smartphone 10, as well as containing a blood glucose meter. In this embodiment, the protective case opens up, via a hinged cover, a slide-open panel, or similar container, to reveal the glucose meter and testing supplies. The container may include: the sensor, test strips, a lancet, logic, a processor, a transmitter, or other components typically found in blood glucose meters. Therefore, the cover provides a self-contained blood glucose test system that attaches to an electronic device. Although this disclosure focuses on a smartphone, this invention is intended to extend to covers and cases for any type of electronic device, including: phones, tablets, music players, laptops, or any other type of device that is later arising. One objective of the case may be to cover or protect the electronic device while being sleek and compact. With regards to a smartphone, it may be an objective to attach to the rear of the smartphone on all four sides, while remaining sleek, such that both the smartphone and the case both remain compact and easy to transport by the user. For example, a smartphone designed to be held in a pocket should still be able to fit in a pocket with the case attached.

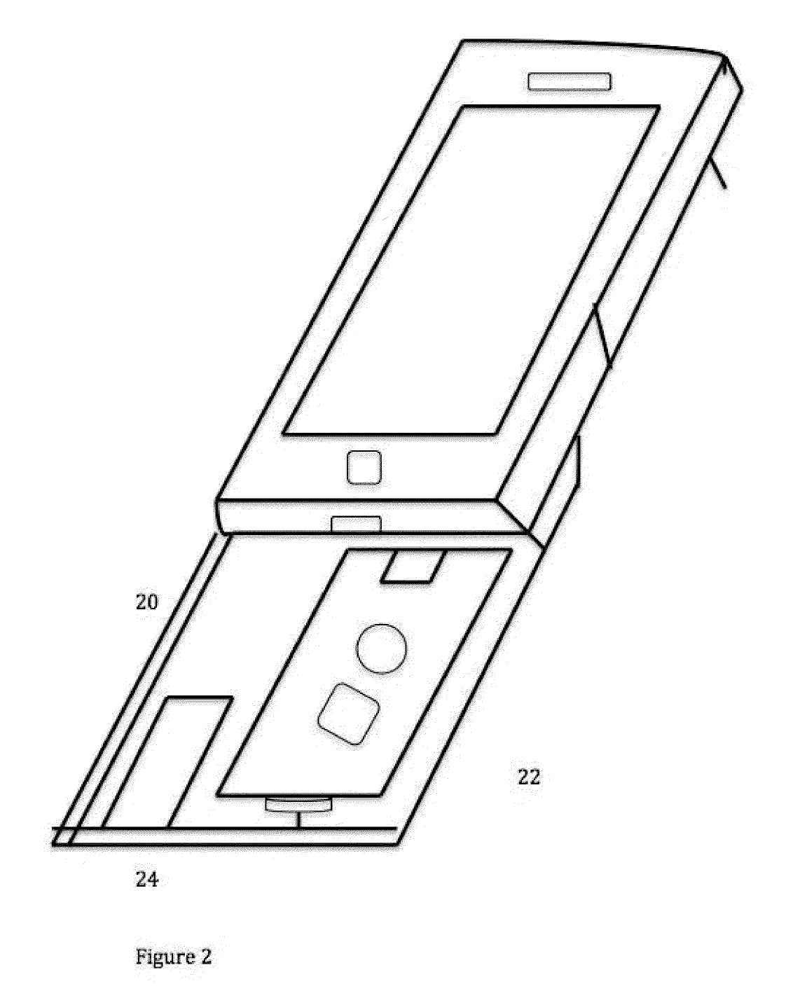

As shown in FIG. 2, the protective case may slide down from the rear of the case. Therefore, the structure of the case 12 attaches to the smartphone 10 on all four sides of the smartphone. And, a rear cover 20 attaches to the rear surface of the protective case 12 to enclose a concealed internal volume in the case 12. FIG. 2 shows a blood glucose meter 22 and test strips 24 held by the rear cover.

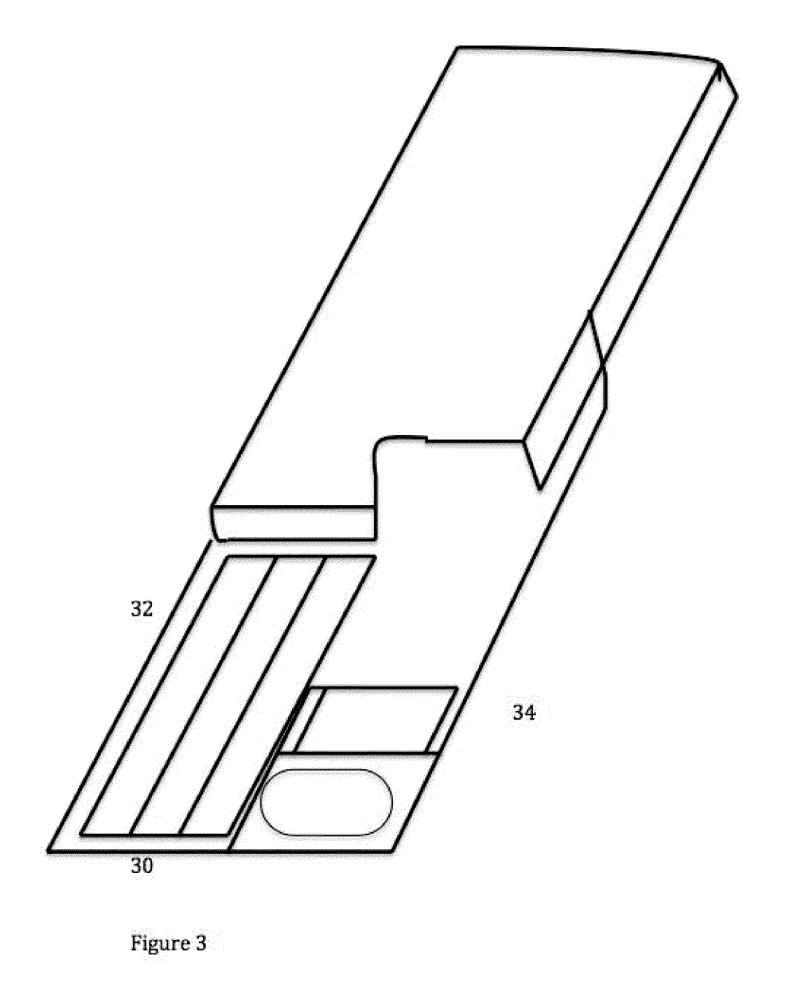

FIG. 3 shows the reverse view of the case 12, with the rear cover 20 slid downwards. This view shows the view of the case structure 30 that is attached to the rear of the smartphone 10. This reveals another side of the concealed volume. On this side of the volume, the case may contain insulin injectors 32. This also shows the circuits of the case 34, such as a processor, wireless communications module, or any other logic incorporated into the case.

In one embodiment of the invention, the entire test system, including the sensor 22, processor 34, and logic 34, are all contained within the protective cover 12. In this embodiment, only the test results may be communicated to the electronic device. However, in an alternate embodiment, the protective cover contains only part of the glucose meter electronics—such as the sensor itself. In this embodiment, only the sensor data may be communicated to the smartphone. Then, a processor in the smartphone may be used to process the sensor data into a final, readable result that is delivered to the end user. In another embodiment, it may also be possible for some of the processor functions or logic to be performed by the processor or software of the smartphone.

In a variation on the protective case, the case may house a self-contained “insert” or holding structure that contains all of the necessary components for the glucose meter. This insert can be common to all type of phones and devices. Then, the smartphone protective case can simply be designed to fit a particular model, with an internal space sized to hold the insert. In one variation, all the electronics may be contained on the insert, but in another variation, the insert may hold the physical aspects of the system, such as the lancet and test strips, while the electronics or sensor are contained in the case. Lancet here means a device for exposing or extracting blood. Any various allocation of system components between the insert and the case are possible and within the scope of this invention. Within the scope of this embodiment, the insert may be housed within a compartment of the case. However, it may also simply slide or snap into the protective case for easy removal. Various types of attachments between the insert and the case known in the art are applicable to this invention and may be used.

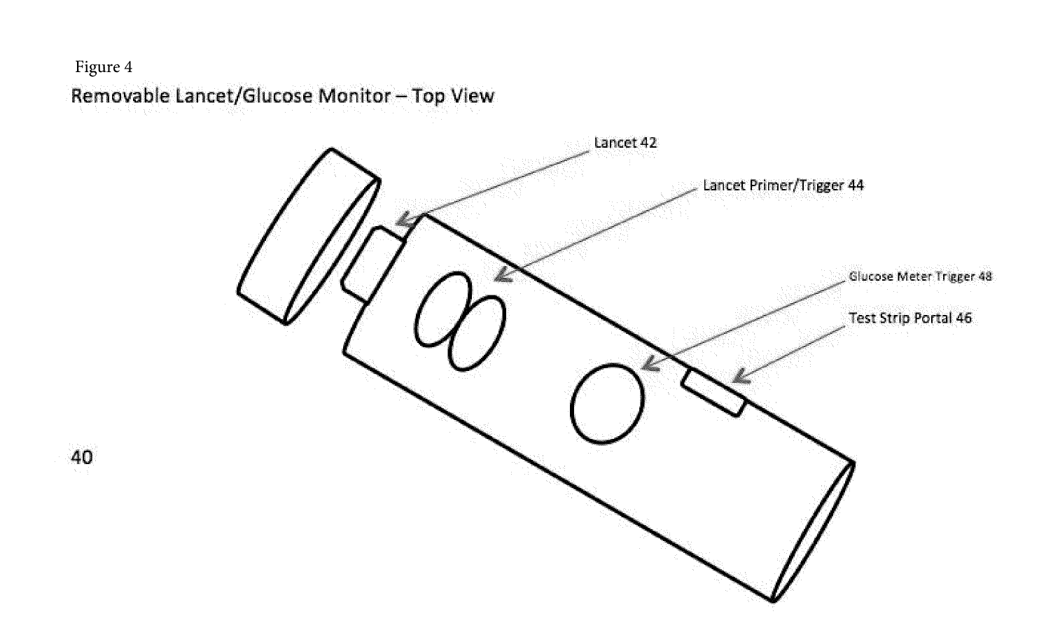

FIG. 4 shows an embodiment of the glucose case, in which the lancet 42 and tester may be placed in one insert unit 40. For example, the one generic insert described above may be snapped in any case that is adapted to particular phones. Then, one may remove the test unit 40 from the backside of the case for blood testing. Once removed from the case, the insert 40 may be removed from the case. The user may push lancet trigger 44 to cause lancet 42 to expose the user's blood. Then the user may place the blood on a test strip and insert the strip into the unit at the test strip portal 46. Then one may press the glucose meter trigger 48 to trigger the test. Then the unit 40 can be reinserted into the case.

There may be several variations on the insert embodiment. For example, the insert may be placed into a space in the case. The case may have a structure attached to the phone, as well as a rear cover. Then, the insert may be placed into that space and the rear cover closed—either via a slide or a hinge, among other design options. However, in another embodiment, there may be no rear cover, and the insert 40 may attached directly into the case. There, a case would attach to the phone, and the insert could be snapped off of the rear of the case, slid into the case, or fastened by any other means known in the art. An advantage of this embodiment is that the case could be adapted to a particular phone, but the insert may be generic across all electronic devices.

Yet another embodiment would put more of the functionality on the insert and less functionality on the case structure. In FIG. 4 above, the lancet and glucose test system is place on the generic insert 40. The test strips could also be placed on the insert. An insulin injector could be placed on the insert. In another embodiment, all electronic functionality of the system—including the integrator, communications module, processor, or any other functionality—could be designed into the insert unit 40. That would leave the case structure attached to the phone as simply structural—simply to attach to the phone and to hold the insert unit in place.

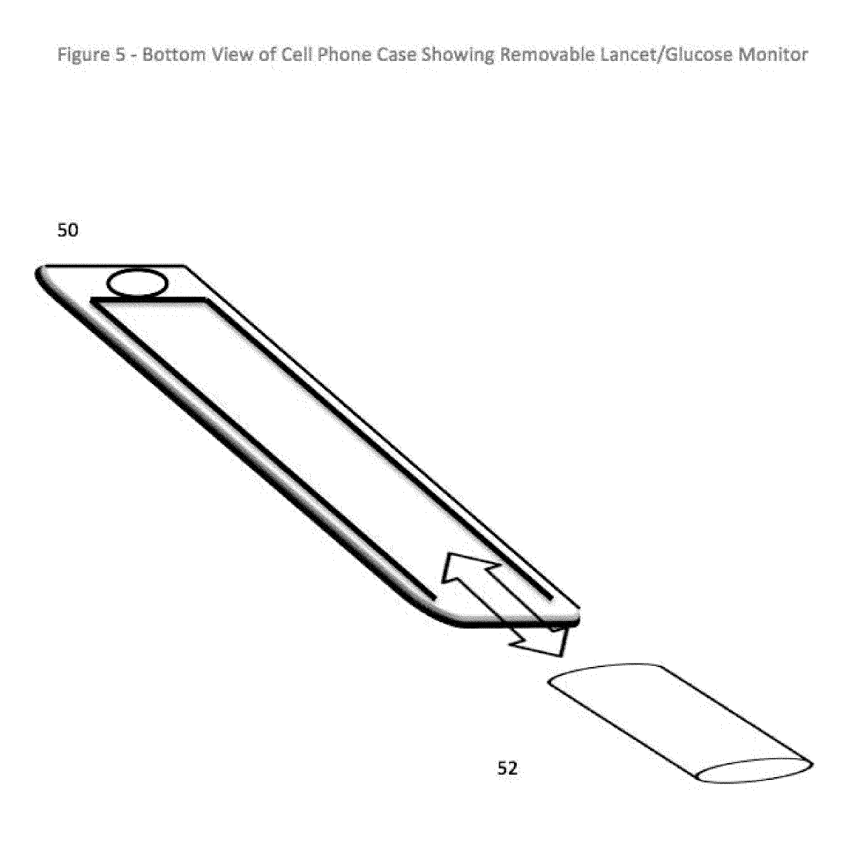

In addition, as shown in FIG. 5, the visible surface of the insert unit 52 may have a protective or decorative surface on it—serving as a rear cover in itself, as opposed to a separate rear cover. In this embodiment, the insert unit 52 may contain any combination of functionality described above, then slide into the case structure 50, where the case structure 50 is attached to the electronic device. For the purposes of this application, when the case structure 50 “contains” the insert unit 52, it could be that 52 is completely contained inside a space enclose by a separate rear cover, or it could simply mean that insert unit 52 is attached to the rear surface of the case structure 50 in some way. A battery may be placed in either part of the case to power the system, and more than one may be used.

Accordingly, there are two types of software applications that may be present on the electronic device to work with any of the above embodiments. In one embodiment, the smartphone may have a fairly simple app on it that may simply take the final, processed, end result of the glucose meter and communicate that to the user of the smartphone via the screen. However, in the case where the processing logic of the smartphone is used to manipulate raw sensor data, the application on the smartphone may have further capability to take sensor data from the cover, and manipulate it via the processing logic within the smartphone—then communicate those results to the end user. In either case, the software application may optionally have other features, such as an alarm to remind the smartphone user when it is time to test again, to predict when glucose level may reach a critical level in the future, to alert the user when it is time to inject, to store old test data in memory, or to communicate test data to a remote location using the wireless communication functions of the smartphone. In another variation of the application, the software may store blood glucose results recorded over time. In another variation, the software may be configured to communicate the results over a wireless connection (802.11x Wi-Fi or cellular network) to a destination, such as a health care provider or online database. In yet another variation, the entire application or results storage may be resident remotely on a cloud.

In a typical embodiment of the system, a blood glucose sensor 22 will reside within the protective smartphone case 20. When used, this sensor will send its signal to a processor which is interpret the sensor signal and convert or translate that into a digital signal relating to blood glucose level, which can be communicated to the user of the device. This signal may also be sent to a transmission circuit—either wired or wireless—which may transmit the blood glucose reading to the smartphone for display to the end user or further transmission via the smartphone to another user over a network.

In another embodiment, there may be no interface with the smartphone at all. In that embodiment, the entire blood glucose meter, including a final measurement reading or result that is visible to the end user may be contained within the protective cover without any reliance on the smartphone.

Turning to another embodiment, it is possible to integrate the glucose meter system into the back cover of a smartphone. For example, some smartphones have replaceable covers for customization or to attach a charger, for example. Here, the cover may be replaced with the portable glucose meter system. That may be advantageous to interface directly with connectors on the smartphone without extraneous wires. The cover may also provide additional material to provide protection, as well, in addition to being cosmetic. In that case, the cover would attach to existing cover connection points on the backside of the phone. The cover may need to be individually tailored to specific phone models.

In yet another embodiment, the smartphone system described above may be attached with one or more insulin syringes 32. Therefore, if a user of the system receives information that they need to inject, he or she may remove an insulin syringe from a compartment in the smartphone case. Then, the user may inject insulin from the syringe 32. As the attached figures show, one or more small syringes 32 may be hidden within a compartment of the smartphone case 30. However, any design of attachment of a syringe to the meter system would accomplish the same function. In addition, such a syringe compartment in a smartphone case could be combined variously with the different versions and aspects of the system described above, and still accomplish the goals or functions of the system. The syringes could be concealed into a smartphone case without the meter system above, also. It may further be desirable to provide syringes of different dosages for different blood glucose readings. In yet another embodiment, a syringe could be loaded by the smartphone glucose meter and injection system with a given dose of insulin based on a reading taken by the meter. For example, a user may take a glucose reading, then the smartphone case may load a syringe with a corresponding dose of insulin required in response to the reading. This could be accomplished with an internal vial of insulin, a pump, and an empty syringe. However, there are a variety of ways to implement such a system while accomplishing the same result.

Any combination of the above features and options could be combined into a wide variety of embodiments. It is, therefore, apparent that there is provided in accordance with the present disclosure, systems and methods for designing and using portable glucose meter systems. While this invention has been described in conjunction with a number of embodiments, it is evident that many alternatives, modifications, and variations would be, or are apparent to, those of ordinary skill in the applicable arts. Accordingly, applicants intend to embrace all such alternatives, modifications, equivalents and variations that are within the spirit and scope of this invention.

What is claimed is:

1. A case for attachment to an electronic device, such as a smartphone or tablet, the case comprising:
a case adapted to attach to the rear surface of an electronic device, said structure comprising:
a structure to interface with the rear surface of an electronic device,
surfaces protruding from said structure and perpendicular to the largest plane of said electronic device that surround at least a portion of each of the long edges of said electronic device, and
a provision for containing a blood testing module in said structure;
a blood testing module configured to test and analyze a blood sample; and
a wireless communication module configured to communicate the results of said analysis of said blood sample to the electronic device.

2. The case of claim 1, wherein said blood testing module comprises:
at least one lancet;
at least one test strip;
a processor;
a testing module configured to analyze blood placed on said test strip when said test strip is brought into contact with said testing module.

3. The case of claim 2, wherein said blood testing module is adapted to test blood glucose levels.

4. The case of claim 3, wherein said lancet and said testing module are placed on a separate insert that is contained in said case structure.

5. The case of claim 4, further comprising at least one insulin injector.

6. The case of claim 1, wherein at least part of said blood testing module uses the software on the electronic device to perform analysis functions.

7. The case of claim 3, wherein said case is sized to be portable and carry upon one's person when attached to a smartphone.

8. The case of claim 1, wherein said wireless communication module uses the Bluetooth communication protocol.

9. The case of claim 1, further comprising a software module resident on the electronic device, wherein said software module communicates with said case and records a history of results from said blood testing module.

10. The case of claim 1, further comprising a software module resident on the electronic device, wherein said software module communicates with said case and notifies the user of the electronic device when it is time for an injection.

11. The case of claim 1, further comprising a software module resident on the electronic device, wherein said software module communicates with said case and notifies the user of said electronic device when it is time to test their blood.

12. The case of claim 1, further comprising a software module resident on the electronic device, wherein said software module communicates the results of said blood testing module to a remote location using a wireless communication circuit in the electronic device.

13. The case of claim 4, further comprising a software module resident on the electronic device, wherein said software module communicates with said case to record a history of results from said blood testing module, notify the user of said electronic device when it is time for an injection, notify the user of said electronic device when it is time to test their blood, and to communicate the result of said blood testing module to a remote location using a wireless communication circuit in the electronic device.

14. The case of claim 5, further comprising a software module resident on the electronic device, wherein said software module communicates with said case to record a history of results from said blood testing module, notify the user of said electronic device when it is time for an injection, notify the user of said electronic device when it is time to test their blood, and to communicate the result of said blood testing module to a remote location using a wireless communication circuit in the electronic device.

15. The case of claim 7, further comprising a software module resident on the electronic device, wherein said software module communicates with said case to record a history of results from said blood testing module, notify the user of said electronic device when it is time for an injection, notify the user of said electronic device when it is time to test their blood, and to communicate the result of said blood testing module to a remote location using a wireless communication circuit in the electronic device.

16. A system for testing blood glucose with a smartphone, the system comprising:
a case adapted to attach to the rear surface of a smartphone, said case comprising:
a structure to interface with the rear surface of an electronic device,
surfaces protruding from said structure and perpendicular to the largest plane of said smartphone that surround each of the long edges of said electronic device smartphone, and
a provision for containing an insert in said structure;
a processor;
a blood glucose testing system in communication with said processor, configured to test and report the glucose level of a blood sample; said blood glucose testing system comprising:
at least one lancet,
at least one test strip, and
a testing module configured to analyze blood placed on said test strip when said test strip is brought into contact with said testing module;
a wireless communication module in communication with said processor configured to communicate the results of said analysis of said blood sample to said smartphone;
a software module resident on said smartphone comprising:
a module designed to communicate with said case via said wireless communication module;
a module designed to receive a report of said glucose level from said blood glucose testing system, and
a module designed to alert a user of said system if they need to inject insulin; and
at least one insulin injector contained in said structure of said case.

17. The system of claim 16, wherein said wireless communication module uses the Bluetooth protocol.

18. The system of claim 17, wherein said software module further comprises:
a module designed to record a history of results from said blood testing system, a module designed to notify the user of said system when it is time to test their blood, and
a module designed to communicate the result of said blood testing system to a remote location using a wireless communication circuit in said smartphone.

19. The system of claim 17, wherein said case is sized compact enough to keep the joined smartphone and case system portable on the user's person.

20. The system of claim 17, wherein said insert structure contains said lancet and said testing module, wherein said insert attached to said case structure.
